(19) United States
(12) Patent Application Publication
(10) Pub. No.: US 2021/0304853 A1
(43) Pub. Date: Sep. 30, 2021

(54) VALIDATING INTERPRETABILITY OF QSAR AND QSPR MODELS

(71) Applicant: Genentech, Inc., South San Francisco, CA (US)

(72) Inventors: Kim BRANSON, Emerald Hills, CA (US); Cuong Quoc NGUYEN, South San Francisco, CA (US)

(73) Assignee: Genentech, Inc., South San Francisco, CA (US)

(21) Appl. No.: 17/217,867

(22) Filed: Mar. 30, 2021

Related U.S. Application Data
(60) Provisional application No. 63/003,054, filed on Mar. 31, 2020.

(51) Int. Cl. G16C 20/70; G16C 20/40

(52) U.S. Cl. CPC G16C 20/70; G16C 20/40

(57) ABSTRACT

A method performed on a computer or computing system, the method comprising steps for aiding interpretability of calculated values in the context of molecular structural features. The method starts with a machine learning model of a pharmacokinetic or physicochemical property of a molecule, derived from a training set of molecules, and provides a user with an interpretability model of the machine learning model for a set of molecules of interest.

### CLAIM OF PRIORITY

This application claims the benefit of priority under 35 U.S.C. § 119(e) to U.S. provisional application Ser. No. 63/003,054, filed Mar. 31, 2020, which is incorporated herein by reference in its entirety.

### TECHNICAL FIELD

The technology described herein generally relates to methods for calculating a pharmacokinetic property or a physicochemical property such as a partition coefficient for an organic molecule, and more particularly relates to applying mathematical methods for aiding interpretability of calculated values in the context of molecular structural features.

### BACKGROUND

An important step in the development of new drugs is the identification of compounds, not previously tested against a particular biological target, but which may share important properties or structural features in common with a molecule of known activity.

Pharmaceutical company compound databases, stored on digital computers, can be enormous, often containing structural and physicochemical data for many millions of compounds. The use of combinatorial synthesis procedures can also lead to very large databases of molecules having specifically tailored properties and/or based on a common scaffold. It has also become realistic to generate databases of ‘virtual’ compounds, i.e., molecular structures of compounds that have never actually been synthesized but whose assembled structural properties can be compared with those that have. In fact, today, chemists have at their fingertips the ability to synthesize—on demand—at least billions (10⁹) of compounds, even though the majority of those compounds will not be tested or studied in detail.

The result is that, even for many of the molecules that have actually been made, reliable data for many key physicochemical parameters does not exist. Consequently, there is a huge gulf between molecules for which reliable experimental data is at hand and those molecules for which there is no such available data but which command legitimate scientific interest meaning that—correspondingly—there is an urgent need for such data.

During the course of a successful drug development program, the path towards a candidate molecule suitable for enduring the rigors of clinical testing will have been built up by establishing relationships between many families of structurally similar compounds. Understanding how the underlying structural variations contribute to improvements or degradations of a physical property (measured on a whole-molecule basis) within a family is key to informing chemists how to improve their molecular designs.

Certain physicochemical properties are critical in determining whether a given molecule is likely to be a good candidate for research, testing, and development as a pharmaceutical. Properties such as Log P (the octanol/water partition coefficient) and Log D (the distribution coefficient), which both act as surrogates for “lipophilicity”, or an indicator of “hydrophobicity”, are understood to be very reliable predictors of pharmaceutical penetration to their targeted sites of physiological action. Correspondingly, many pharmacokinetic parameters (often referred to as “ADMET” in shorthand) are hard to measure and difficult to systematically predict. Furthermore, actual values of such properties are only known reliably for relatively few molecules and are not trivial to measure. Therefore, a number of computational methods for predicting properties such as these have been developed. Predictions rely on models that have been developed based on known (measured) molecular data. Most models attempt to dissect a given property of a molecule into specific contributions from its constituent atoms or functional groups. To the extent those contributions are transferable, then predictions can be made for other molecules whose structures share those particular atoms or groups.

Other ways of obtaining reliable values of a property such as Log D involve utilizing techniques such as machine learning on datasets of known values. It is possible to predict a value of a property for many molecules in this way, but the challenge is that a machine learning model offers very little in the way of explanation behind its calculations. Consequently there can be a lack of confidence in the results of using such methods.

Currently absent from many models of both physicochemical and pharmacokinetic properties, therefore, is an aspect of interpretability: that is to say, many chemists who are deeply involved in designing novel molecules are looking for greater levels of insight from computational models than simply a prediction of a numerical value for a single molecule with known accuracy. Most chemists think not of isolated data points but in terms of structure-activity relationships (SAR's), whether through mappings of actual biological activity to structural features (as in quantitative structure activity relationships—QSAR's) or through mappings of specific properties to structure (as in quantitative structure property relationships—QSPR's).

Accordingly, there is a need for a method of building a model of a physicochemical or pharmacokinetic property that can be both reliable and interpretative.

The discussion of the background herein is included to explain the context of the technology. This is not to be taken as an admission that any of the material referred to was published, known, or part of the common general knowledge as at the priority date of any of the claims found appended hereto.

Throughout the description and claims of the instant application the word “comprise” and variations thereof, such as “comprising” and “comprises”, is not intended to exclude other additives, components, integers or steps.

### SUMMARY

The instant disclosure addresses the processing of machine learning models of molecular property data. In particular, the disclosure comprises a computer-implemented method or process for building an interpretability model of a machine learning model. The disclosure further comprises a computing apparatus for performing the methods described herein. The apparatus and process of the present disclosure are particularly applicable to property prediction and model building for physicochemical and pharmacokinetic properties of relevance to development of commercially and clinically viable pharmaceuticals.

In one aspect, the method comprises: receiving test molecular structure data for a test molecule, wherein the molecular structure data for the test molecule comprises an atom type for each atom in the test molecule; inputting the test molecular structure data into a global model of a physicochemical property, wherein the global model comprises a contribution of each of a plurality of atom types to a value of the physicochemical property for the molecule, and wherein the global model was trained using a set of training molecules for which the value of the physicochemical property was known from experimental measurement; generating a local model of the physicochemical property, wherein the local model is based on molecules in the neighborhood of the test molecule and wherein the neighborhood is defined according to a threshold value of a similarity metric; optimizing the local model according to one or more best-fit criteria; validating the best-fit local model by: using a match-pairs analysis to establish a set of molecules related to the test molecule by a set of respective chemical transformations; obtaining from the best-fit local model weighted contributions to the physicochemical property of atoms and functional groups in the test molecule and atoms and functional groups in the set of molecules related to the test molecule; for each molecule in the set of molecules related to the test molecule, calculating a first delta, wherein the first delta is the difference between the value of the sum of the weighted contributions of the one or more atoms in the chemical transformation of the molecule and value of the sum of the weighted contributions of the one or more atoms in the chemical transformation for the test molecule; for each molecule in the set of molecules related to the test molecule, calculating a second delta, wherein the second delta is the difference between the value of the physicochemical property calculated in the global model for the molecule and the value of the physicochemical property calculated in the global model for the test molecule; and deriving from the values of the first delta and the values of the second delta the validity of an interpretability model for the physicochemical property wherein the interpretability model comprises weighted contributions of atoms and functional groups for a molecule in the set of molecules related to the test molecule to the value of the physicochemical property for the molecule.

The validity of the interpretability model that is a result of the process aids pharmaceutical and computational chemists, for example, in assessing their confidence in the machine learning model for the physicochemical property.

### BRIEF DESCRIPTION OF THE DRAWINGS

FIG. 1 shows a schematic of the principles underlying the LIME method as applied to a general function, f(x);

FIG. 2 shows a schematic of an exemplary computing apparatus for performing a process as described herein;

FIG. 3 shows graphical representations of atomic contributions to Log D for three molecules;

FIGS. 4A, 4B, and 4C show a case study of the methods herein, as applied to Log D for benzene derivatives; and

FIG. 5 shows results from a validation data set of the methods described herein.

### DETAILED DESCRIPTION

The instant technology is directed to methods of creating an interpretability model for a pharmacokinetic or physicochemical property such as, but not limited to, Log P or Log D. The methodology and examples herein are described with respect to Log P or Log D, but it would be understood by one of skill in the art that the methodology could also be applied to some other physicochemical property, or to a pharmacokinetic property, for which a machine learning model can be built. Representative pharmacokinetic properties include, but are not limited to, those that are important in assessing a molecule's viability to become a clinically successful pharmaceutical, for example, adsorption, distribution, metabolism, excretion, and toxicity (often referred to collectively as “ADMET”). It would be equally apparent to one skilled in the art that other complex and specific physiological properties could be modeled in a comparable manner. Such properties can include aspects of pharmaceutical behavior such as brain penetrability, or kinetic solubility. It is equally possible to use the methods herein to model combinations of two or more properties, such as kinetic solubility and Log D.

### Log P and Log D

A partition coefficient (P) or distribution coefficient (D) represents a quantitative comparison of the solubilities of a solute in two immiscible solvents. Such a coefficient is defined as the ratio of equilibrium concentrations of the compound in the mixture of the two liquids. Given the wide range of possible values of such a coefficient (covering many orders of magnitude), it is invariably represented on a log scale.

As generally one of the two solvents utilized is polar (such as water), whereas the other is non-polar, the partition coefficient is most usefully applied in the case of compounds that do not ionize. It is therefore understood that Log P refers to the logarithm of the concentration ratio of un-ionized species of the compound. Conversely, as most pharmaceutically active molecules of interest are ionized in aqueous solution, the distribution coefficient is most often used in pharmaceutical applications and refers to the concentration ratio of all species of the compound (ionized plus un-ionized). Log D is the same as Log P for molecules that do not ionize; but for compounds that do ionize, there is a pH-dependence of values of Log D.

In the chemical and pharmaceutical sciences, Log P refers to the partition between water and 1-octanol. Thus, Log P measures the hydrophobicity of a molecule and is useful in estimating how effectively a drug molecule is likely to distribute within the body. Hydrophobic drugs with high Log P are readily located in hydrophobic areas such as lipid bilayers of cells, whereas hydrophilic (non-hydrophobic) drugs most easily stay in aqueous regions. The challenge in pharmaceutical design is to balance the desire to see the drug have sufficient hydrophobicity to distribute within the body versus a tendency of more hydrophobic molecules to be retained for longer, with possible toxic or other adverse consequences.

The fact that an actual value for Log P (or Log D) for a molecule must be determined through an experimental measurement, yet medicinal chemists need to be able to make rapid decisions about the potential viability of dozens or hundreds of compounds before committing resources to a development program, means that reliable predictors of Log P (that thereby obviate the need to carry out an experimental measurement) have become a mainstay of pharmaceutical research.

The majority of predictors of Log P are parameterized: known experimental values across a range of compounds are dissected into contributions to each molecule's total (measured) value from its constituent atoms or fragments, on the assumption that an equivalently situated atom or functional group contributes in the same way to any given molecule.

While some atomic parameter sets have been well established yet have unsatisfactory performance, particularly for novel molecules, the more recent application of machine learning methods such as neural networks to problems of molecular property prediction have brought with them additional challenges for chemists. Such methods produce models of the property in question that can be progressively refined as more data is acquired but are designed to be applicable to any molecule for which a calculated value of the property is needed.

Despite the apparent simplicity of the implementation and output of such models, it remains difficult in many instances to understand a prediction for a given molecule (particularly in comparison to other similar molecules), and, correspondingly to achieve a requisite level of trust in the model as a whole. A model of a property such as Log P could be more readily accepted by its user base if each predicted value for a molecule were accompanied by an aid to interpretation. Such an aspect of interpretability would provide insight into the usefulness of the model and may yet guide ways to improve it.

### Local Interpretability Models

A method referred to as a Local Interpretable Model-Agnostic Explanation (LIME) (see, e.g., the document at Internet site arxiv.org/pdf/1602.04938.pdf, incorporated herein by reference) is applied herein to predictions of molecular properties such as Log P and Log D.

The fundamental idea of LIME is that, when looking at a small enough region of any function, regardless of its complexity, it appears to be linear or almost linear within the interval considered. Given a trained model and a new instance, LIME proposes building a simple and explainable model (called an explainer) that is faithful locally (but not necessarily globally) to the trained model.

In practice, assuming that there is already a trained model, f(x), the steps are as follows:

Given a new instance, denoted molecule x, we sample a vector of N molecules, X′={x′₁, x′₂, . . . x′_{N}}, from the neighborhood of x.

Next, we compute a trained model prediction on the neighborhood sample f(X′).

We then compute the similarity of the neighborhood samples to x, denoted sim(x,X′).

The training set, D, for the explainer f′(x) is:

D={x,f(x),1}U{x′ᵢ,f(x′ᵢ),sim(x,x′ᵢ)} (1)

Now, f′(x) can be trained on D with samples weighted by similarity. The training can be carried out by a simple algorithm such as linear regression or least squares. The weights of f′(x) provide feature importance.

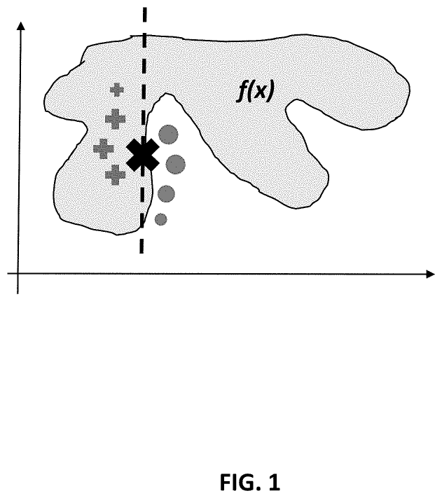

FIG. 1 provides a schematic of this process. In FIG. 1, f(x) is a complicated function represented as a projection on to an orthogonal two-dimensional axis system. In FIG. 1, the vertical dashed line is the explainer In the local region of “X”. The plus signs and filled circles on either side of the dashed line are the values of f(x) for molecules in the neighborhood of X.

### Matched Molecular Pairs

The method of matched molecule pairs (see, e.g., Griffen, et al., J. Med. Chem., (2011), 54, 7739-7750) provides a convenient tool for defining a similarity space around a molecule of interest. Those molecules that differ from the molecule of interest by single chemical transformations (such as an atom—atom substitution, an atom—group substitution, inserting a single atom, inserting a functional group, or a group—group substitution) can be quantified and used to calibrate the calculation of differences between values of the physicochemical property for pairs of molecules. The method is predicated on the principle that it is easier and more reliable to calculate a difference (a “delta”) between the values of a property for two molecules that differ from one another by a small change than it is to calculate absolute values of that property for each of the two molecules independently. Conversely, by identifying a common chemical transformation that applies to several pairs of molecules, the constancy of the contribution of that chemical transformation across large numbers of molecules can be explored.

### Molecular Structural Representations

Two-dimensional (“2D”, or “2-D”) structure diagrams can be considered to be the “natural language” of chemists, not least because this graphical representation of structures allows molecules to be instantly appreciated in ways that a systematic name does not afford. A 2-D representation of a molecule relies solely on defining the atoms present (carbon, hydrogen, oxygen, etc.) and the types of covalent bonds they make with one another. Absolute spatial coordinates that define an actual 3-dimensional conformation of a molecule are largely irrelevant to both the 2-D representation and a chemist's appreciation of the molecule's identity.

Although the development of sophisticated computer graphics programs over the last several decades has made it easy to display and manipulate three-dimensional (“3D” or “3-D”) structures of molecules, there remains sufficient information in a 2-D representation for efficient and valuable predictions of molecular properties to be made.

### Methodology

In overview, the present technology includes a method, comprising at least in part the following steps as performed on a computer system as further described herein. This the technology includes a computer-implemented method that comprises the following steps.

The computer system receives test molecular structure data for a test molecule, wherein the molecular structure data for the test molecule comprises an atom type for each atom in the test molecule. By atom type is meant a descriptor that can be unambiguously applied to any atom based on its element type and location in a molecular structure. At its simplest, an atom type may simply be the element type (C, O, H, N, etc.), in which case all carbon atoms would be considered equivalently regardless of which atoms they bond to in the molecule. More useful sets of atom types discriminate according to successively distant neighborhoods in a molecular structure. Thus, one set of atom types would distinguish carbonyl carbon atoms from saturated (aliphatic) carbon atoms, whereas a more sophisticated one would be able to distinguish carbonyl groups in aldehydes from those in carboxylic acids.

In some embodiments, an atom type for a given atom is represented as a vector of weighted contributions of atoms in the functional group in which the atom is situated. Such a vector can comprise values of properties selected from the group consisting of: atomic number, hybridization (e.g., sp, sp², sp³, as commonly understood by organic chemists), number of neighbors (as typically understood to the number of atoms covalently bonded to a given atom), and aromaticity (as typically understood by organic chemists, a ring in which an atom is situated can be designated aromatic according to attributes such as the number of fully declocalized π-electrons shared by the ring atoms). In such a representation, the vector of weighted contributions for an atom comprises contributions from up to 6 nearby atoms, at least 2 of which are bonded to the atom, the remainder of which are separated from the atom by two, or sometimes more than two, covalent bonds.

It is to be understood that the test molecule is typical of pharmaceutical (“drug”) molecules and other “small organic molecules” found in company databases today. Such molecules typically have from 10-50 non hydrogen atoms, and most typically have from 20-40 non hydrogen atoms. Non hydrogen atoms are atoms other than hydrogen, and are typically selected from two or more of carbon, oxygen, nitrogen, sulfur, phosphorous, and the halogens.

In a preferred embodiment, the molecular structure data is stored and communicated in 2-D form. In other embodiments a 3-D representation may be used for storage even though only the atom type and bond type information is used in a calculation of a physicochemical property using the methods herein. In still other embodiments, the molecular structure data may be stored and/or communicated in a line notation format, such as SMILES.

The test molecular structure data is input into a global model of a physicochemical property, such as Log D or Log P, wherein the global model comprises a contribution of each of the plurality of atom types to a single value of the physicochemical property for the molecule. The global model has preferably been trained using a set of training molecules for which the value of the physicochemical property was known from experimental measurement. The method is not limited to the size of the set of training molecules. The global model is preferably trained on a set of up to 400,000 training molecules, such as up to 250,000 training molecules or up to 100,000 training molecules, where the minimum number in the set of training molecules is typically between 1,000 and 10,000 molecules.

In this way, a value for the physicochemical property for the test molecule can be calculated, within the confines of a pre-existing, understood, global model. The global model is typically one that is based on summing fixed contributions of the various atom types in a molecule to generate a value of the property for the molecule, on the assumption that a given atom type will contribute in the same way regardless of the molecule.

Next, a local model of the physicochemical property is generated, wherein the local model is based on molecules in the neighborhood of the test molecule. In this situation, the neighborhood is defined according to a threshold value of a similarity metric relative to the test molecule. The principle behind generating a local model is to identify a set of molecules that are sufficiently similar to the test molecule that the local model will embody some interpretability to a chemist. The similarity metric utilized to identify these molecules may be any of those known in the art, and preferably one that is based on a 2-D representation of molecular structure that can be condensed into a single number and is easy to compute. In other embodiments, it can be derived from 1-dimensional or 3-dimensional representations of molecular structures. In the case of 3-dimensional representations, the coordinates of the atoms can be obtained from, for example, a crystal structure (say of the isolated molecule or the molecule bound in a protein receptor), or can be obtained from a 3-dimensional structure prediction method. Typically the metric represents an overlap (rather than a distance) and is a number in the range [0,1] and may be based on a Tanimoto coefficient or a cosine metric. Many such metrics exist in the art and have an appeal of simplicity in that the closer the value of the metric to 1.0 the more similar is the pair of molecules under comparison. Furthermore, such metrics also embody an understanding that molecules can be ranked in their similarity to a test molecule according to values of the metric computed for each against that test molecule.

The local model can be optimized according to one or more best-fit criteria. In most model generation, some optimization is necessary and many optimization algorithms known in the art—such as but not limited to least squares fitting, or regression—may be deployed to achieve this for the local model described herein.

Subsequent validation of the best-fit local model may be accomplished in the following way. After using a match-pairs analysis to establish a set of molecules related to the test molecule by a set of respective chemical transformations, it is possible to obtain from the best-fit local model weighted contributions to the physicochemical property of atoms and functional groups in the test molecule and of atoms and functional groups in the set of molecules related to the test molecule. The set of molecules generated through matched pairs analysis need not contain any molecules in common with those that were used to build the local model (i.e., those that are similar to the test molecule according to some similarity criterion).

Now as a way to validate the model, two deltas are calculated.

First, for each molecule in the set of molecules related to the test molecule, a first delta is calculated. The first delta is the difference between the value of the sum of the weighted contributions of the one or more atoms in the chemical transformation of the molecule and the sum of the weighted contributions of the one or more atoms in the chemical transformation for the test molecule.

Second, for each molecule in the set of molecules related to the test molecule, a second delta is calculated, wherein the second delta is the difference between the value of the physicochemical property calculated in the global model for the molecule and the value of the physicochemical property calculated in the global model for the test molecule.

Expressed as a formula, the calculation of the first delta is as follows, for a matched pair of molecules, A and B, such that the transformation of one to the other involves atoms a₁-aₙ and b₁-bₘ respectively:

$$First Δ = {\sum }_{i = 1}^{m} Local {Model}_{B} ( {b}_{i} ) - {\sum }_{i = 1}^{n} Local {Model}_{A} ( {a}_{i} ) \qquad ( 2 )$$

In equation (2), by “LocalModel” is meant “weighted contribution from the local model.”

By way of example, consider two cases of calculating the first delta.

In a first situation, the matched pair involves only removing atoms or a functional group from a reference molecule. For example, in the matched pair, bromobenzene->benzene, the chemical transformation of the matched pair is just removing Br, so:

FirstΔ=null−local_model_bromobenzene(Br)=−local_model_bromobenzene(Br)

In a second situation, the matched pair involves transformation (substitution) of atoms/functional groups. For example, in the matched pair bromobenzene->benzoic acid, the chemical transformation of the matched pair is Br->COOH, so:

FirstΔ=[local_model_benzoic_acid(C)+2×local_model_benzoic_acid(O)+local_model_benzoic_acid(H)]−local_model_bromobenzene(Br).

Finally, from the values of the first delta and the values of the second delta, the validity of an interpretability model for the physicochemical property can be derived, wherein the interpretability model comprises weighted contributions of atoms and functional groups for a molecule in the set of molecules related to the test molecule to the value of the physicochemical property for the molecule. Such deriving can be obtained by, for example, plotting the values of the first delta against the values of the second delta for each of the molecules in the set of molecules related to the test molecule.

For example, to assess the overall validity of the interpretability model, the relationship between the first and second deltas can be measured. Such a measurement can be by the coefficient of determination (R2) or Pearson correlation coefficient between the deltas, where a larger R2 or Pearson correlation means an interpretability model of greater validity. Models with high overall validity can still have low validity for specific transformations, however. Problematic transformations can be identified using outlier detection methods, including but not limited to: local outlier factor, isolation forest, and others known to those skilled in the art. Scientists can then make decisions to exclude such outlier transformations from any analysis using the interpretability model.

### Implementational Details

The methods described herein are preferably implemented on one or more computer systems, and the implementation is within the capability of those skilled in the art of computer programming and/or software development. The functions for carrying out the calculations and numerical computations underlying the methods herein can be implemented in one or more of a number and variety of programming languages including, in some cases, mixed implementations (i.e., relying on separate portions written in different computing languages suitably configured to communicate with one another). For example, the functions, as well as any required scripting functions, can be programmed in one or more of C, C++, Java, JavaScript, VisualBasic, Tcl/Tk, Python, Perl, golang, rust, lisp, .Net languages such as C#, and other equivalent languages. Languages for numerical computation, such as a generation of FORTRAN, may be deployed where suitable. The capability of the technology is not limited by or dependent on the underlying programming language used for implementation or control of access to the basic functions. Alternatively, the functionality can be implemented from higher level functions such as tool-kits that rely on previously developed functions for manipulating chemical structures, and carrying out optimizations.

The technology herein can be developed to run with any of the well-known computer operating systems in use today, as well as others not listed herein. Those operating systems include, but are not limited to: Windows (including variants such as Windows XP, Windows95, Windows2000, Windows Vista, Windows 7, and Windows 8 (including various updates known as Windows 8.1, etc.), and Windows 10, available from Microsoft Corporation); Apple iOS (including variants such as iOS3, iOS4, and iOS5, iOS6, iOS7, iOS8, iOS9, iOS10, iOS11, iOS12, iOS13, iOS14, and intervening updates to the same); Apple Macintosh operating systems such as OS9, OS 10.x, OS X (including variants known as “Leopard”, “Snow Leopard”, “Mountain Lion”, “Lion”, “Tiger”, “Panther”, “Jaguar”, “Puma”, “Cheetah”, “Mavericks”, “Yosemite”, “El Capitan”, “Sierra”, “High Sierra”, “Mojave” and “Catalina”); the UNIX operating system (e.g., Berkeley Standard version) and variants such as IRIX, ULTRIX, and AIX; and the Linux operating system (e.g., available from Red Hat Computing, and other online sources).

To the extent that a given implementation of the technology herein relies on other software components, already implemented, such as functions for generating atom types for a molecular structure, those functions can be assumed to be accessible to a programmer of skill in the art.

Furthermore, it is to be understood that the executable instructions that cause a suitably-programmed computer to execute methods for deriving a local interpretability model, as described herein, can be stored and delivered in any suitable computer-readable format. This can include, but is not limited to, a portable readable drive, such as a large capacity “hard-drive”, or a “pen-drive”, such as can be connected to a computer's USB port, and an internal drive to a computer, and a CD-Rom, or an optical disk. It is further to be understood that while the executable instructions can be stored on a portable computer-readable medium and delivered in such tangible form to a purchaser or user, the executable instructions can be downloaded from a remote location such as a networked server computer (often referred to as “the cloud”) to the user's computer, such as via an Internet connection which itself may rely in part on a wireless technology such as WiFi. Such an aspect of the technology does not imply that the executable instructions take the form of a signal or other non-tangible embodiment. The executable instructions may also be executed as part of a “virtual machine” implementation.

Thus, in sum, the technology herein includes a computer program product that comprises instructions which, when the program is executed by a computer, causes the computer to carry out a method as described herein.

### Computing Apparatus

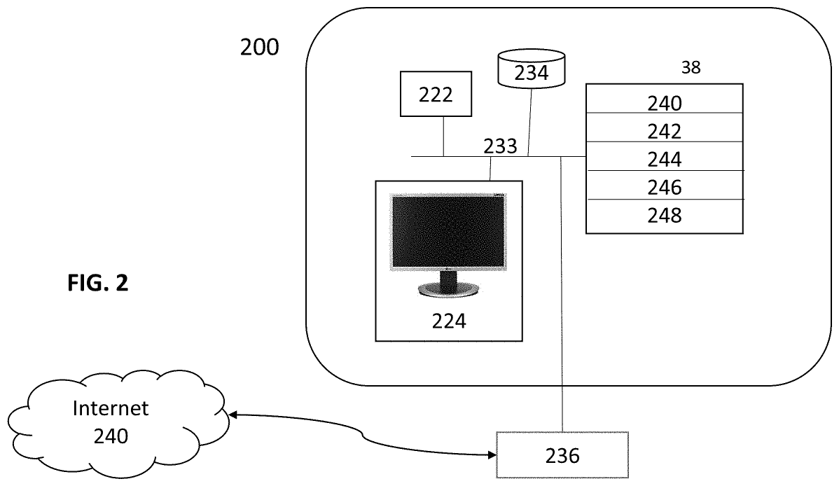

An exemplary general-purpose computing apparatus (200) suitable for practicing methods described herein is depicted schematically in FIG. 2.

The computer system (200) comprises at least one data or central processing unit (CPU) (222), a memory (238), which will typically include both high speed random access memory as well as non-volatile memory (such as one or more magnetic disk drives), a user interface (224), one more disks (234), and at least one network connection (236) or other communication interface for communicating with other computers over a network, including the Internet (240), as well as other devices, such as via a high speed networking cable, or a wireless connection. There may optionally be a firewall (not shown) between the computer (200) and the Internet (240). At least the CPU (222), memory (238), user interface (224), disk (234) and network interface (236), communicate with one another via at least one communication bus (233). Network interface (236) may include both wireless and local area network connectivity.

Memory (238) stores procedures and data, typically including some or all of: an operating system (240) for providing basic system services; one or more application programs, such as a parser routine (242), and a compiler (not shown in FIG. 2), a file system (248), one or more databases (244) that store data such as molecular structures, and optionally a floating point coprocessor where necessary for carrying out high level mathematical operations. The methods of the present technology may also draw upon functions contained in one or more dynamically linked libraries, not shown in FIG. 2, but stored either in memory (238), or on disk (234).

The database and other routines that are shown in FIG. 2 as stored in memory (238) may instead, optionally, be stored on disk (234) if the amount of data in the database is too great to be efficiently stored in memory (238). The database may also instead, or in part, be stored on one or more remote computers that communicate with computer system (200) through network interface (236), according to methods as described in the Examples herein.

Memory (238) is encoded with instructions (246) for at least carrying out the methods described herein. The instructions can further include programmed instructions for performing one or more of: model building, parameter fitting, and optimization. In many embodiments, the model is not calculated on the computer (200) that validates the model but is performed on a different computer (not shown) and, e.g., transferred via network interface (236) to computer (200).

Various implementations of the technology herein can be contemplated, particularly as performed on one or more computing apparatuses (machines that can be programmed to perform arithmetic) of varying complexity, including, without limitation, workstations, PC's, laptops, notebooks, tablets, netbooks, and other mobile computing devices, including cell-phones, mobile phones, and personal digital assistants. The methods herein may further be susceptible to performance on quantum computers. The computing devices can have suitably configured processors, including, without limitation, graphics processors and math coprocessors, for running software that carries out the methods herein. In addition, certain computing functions are typically distributed across more than one computer so that, for example, one computer accepts input and instructions, and a second or additional computers receive the instructions via a network connection and carry out the processing at a remote location, and optionally communicate results or output back to the first computer.

Control of the computing apparatuses can be via a user interface (224), which may comprise a display, mouse, keyboard, and/or other items not shown in FIG. 2, such as a track-pad, track-ball, touch-screen, stylus, speech-recognition device, gesture-recognition technology, human fingerprint reader, or other input such as based on a user's eye-movement, or any subcombination or combination of the foregoing inputs.

The manner of operation of the technology, when reduced to an embodiment as one or more software modules, functions, or subroutines, can be in a batch-mode—as on a stored database of molecular structures, processed in batches—or by interaction with a user who inputs specific instructions for a single molecular structure.

The local interpretability model created by the technology herein can be displayed in tangible form, such as on one or more computer displays, such as a monitor, laptop display, or the screen of a tablet, notebook, netbook, or cellular phone. The model can further be printed to paper form, stored as one or more electronic files in a format for saving on a computer-readable medium or for transferring or sharing between computers, or projected onto a screen of an auditorium such as during a presentation.

Certain default settings can be built in to a computer-implementation, but the user can be given as much choice as he or she desires over the features that are used in calculating the local interpretability model.

In still further embodiments of the technology, a user can interact with the local interpretability model via a touch-screen, to select parts of the model, change display options, select and move portions of a displayed model, or perform other similar operations.

### ToolKit

The technology herein can be implemented in a manner that gives a user access to, and control over, basic functions that provide key elements of display, including but not limited to, the types of graphical elements described herein as well as others that are consistent with principles of representation and display as set forth herein.

A toolkit can be operated via scripting tools, as well as or instead of a graphical user interface that offers touch-screen selection, and/or menu pull-downs, as applicable to the sophistication of the user. The manner of access to the underlying tools by the user is not in any way a limitation on the technology's novelty, inventiveness, or utility.

To the extent that a given implementation relies on other software components, already implemented, such as functions for applying permutation operations, and functions for calculating overlaps of bit-strings, those functions can be assumed to be accessible to a programmer of skill in the art.

### EXAMPLES

### Example 1: Explaining GraphConv Log D Predictions

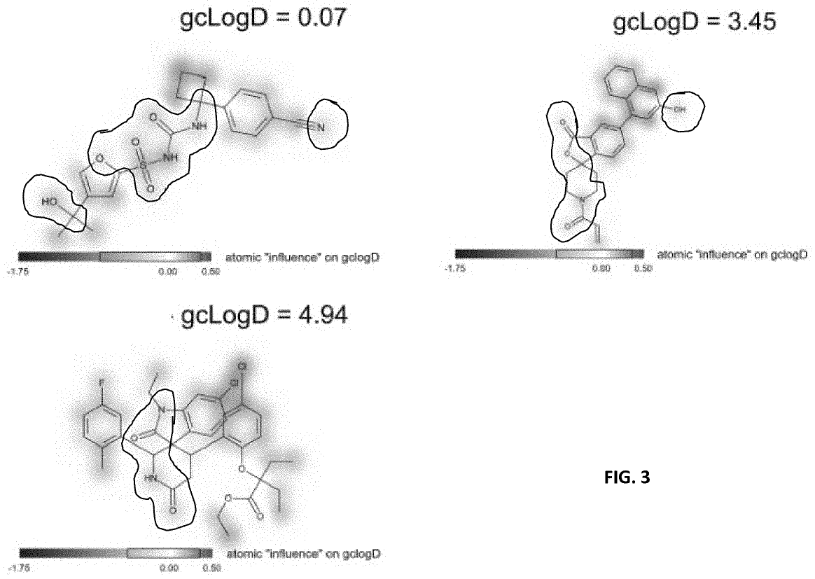

Values of Log D for several molecules, as calculated by the neural network program GraphConv, were analyzed by the methods described herein. (See FIG. 3, in which fragments enclosed in loops are those with negative scores on the spectrum of atomic influence.)

The methods herein were adapted to treat each atom as a feature. As can be seen from the figure, the contributions of each of the atoms are consistent with chemical heuristics.

Negative atoms and groups on the scale correspond to polar groups (hydroxyls, amines, carbonyls, etc.) and electronegative atoms (O, N, S, etc.).

Positive atoms and groups on the scale correspond to non-polar groups such as aromatic and non aromatic cycles, carbon chains.

### Example 2: A Case Study on Benzene Derivatives

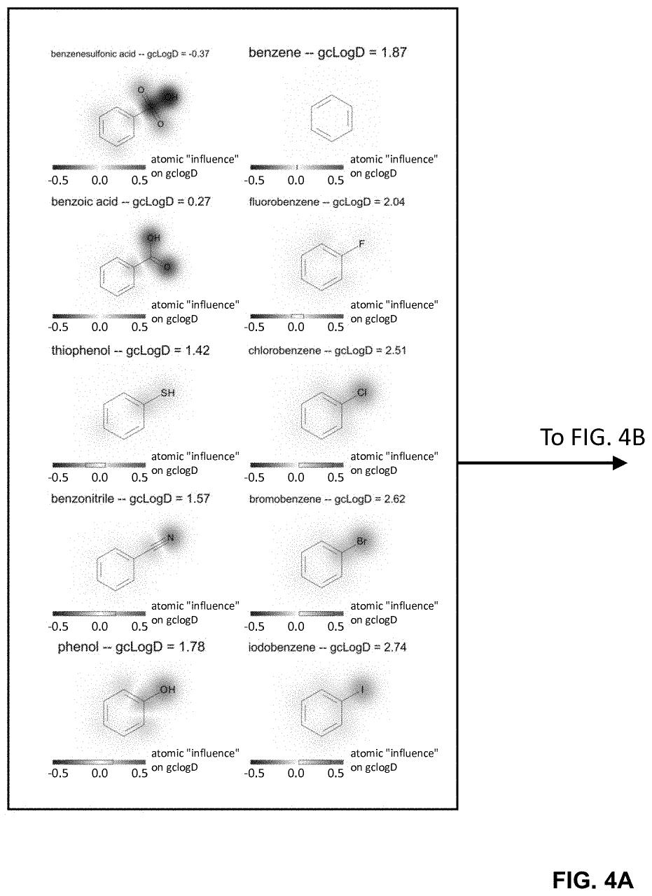

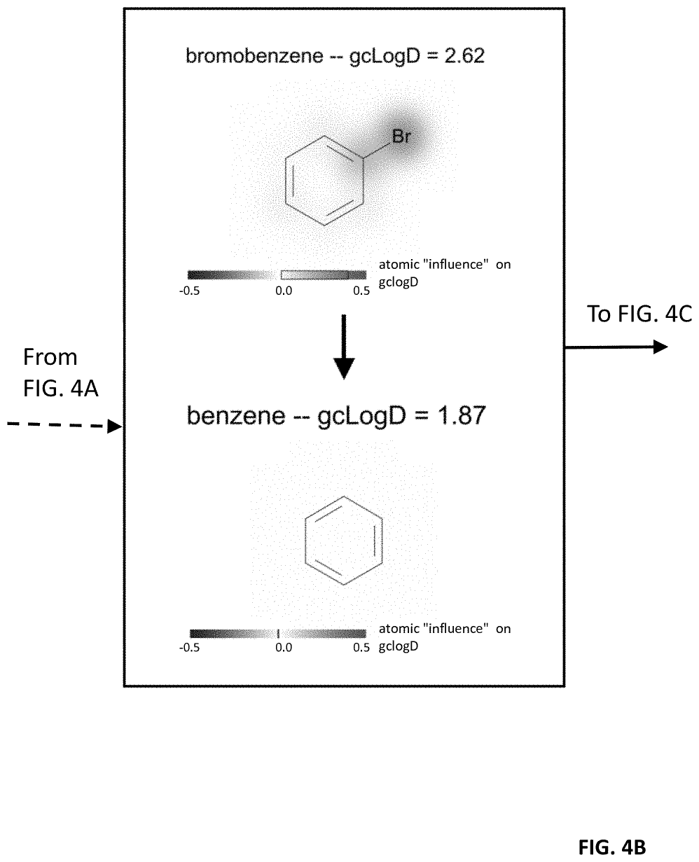

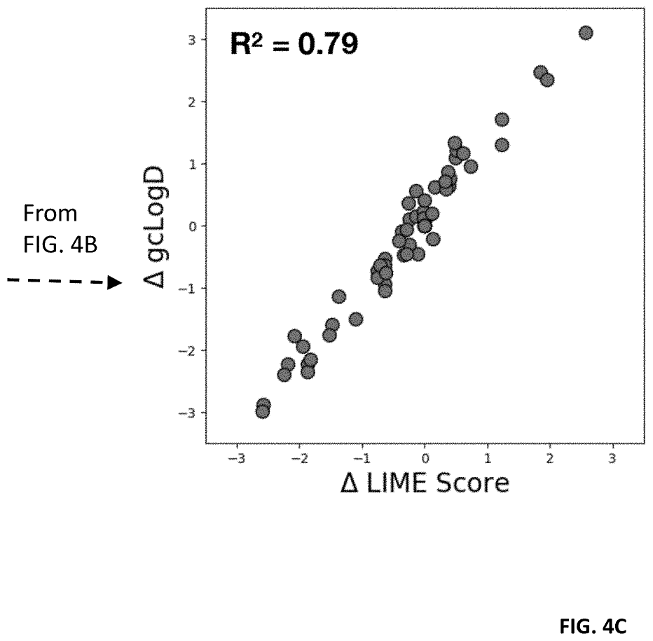

A small scale study of 45 benzene transformations shows that LIME scores extracted from local models accurately represent changes in predictions of a trained GraphConv model. The derivatives and the correlation are shown in FIGS. 4A, 4B, and 4C, in which: a pool of benzene and 9 derivatives (FIG. 4A) is used to get a Δ LIME of substituents and a Δ gc Log D of molecule pairs (FIG. 4B). The plot of the Δ values is shown in FIG. 4C.

### Example 3: Validation Data Set for Log D

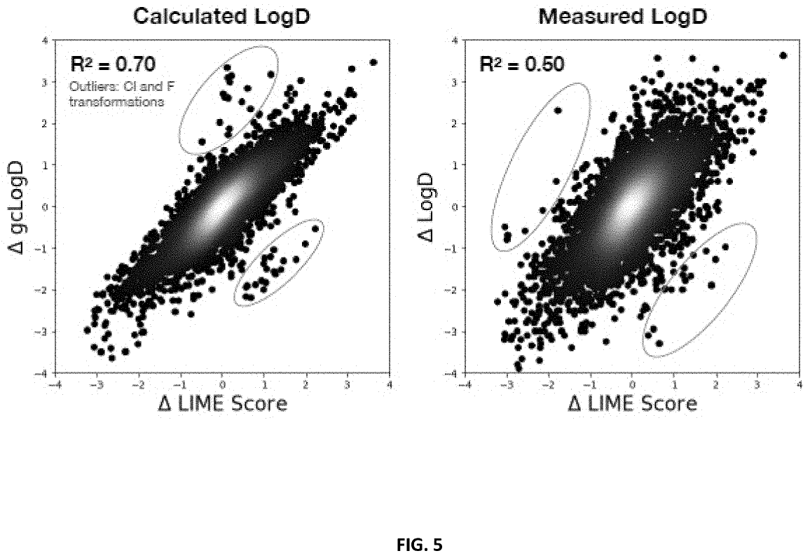

The application of LIME in Example 2 was extended to a larger set of molecules, embodying more complex scaffolds. Validation was carried out on internal matched molecule pairs. Results are shown in FIG. 5.

The graphs are based on 5200 matched pairs from 986 transformations identified by OEMedChem (substituent size <20% of input structures), available from OpenEye Scientific Software, Inc., Santa Fe, N. Mex. From each pair Δ LIME of substituents, Δ calculated Log D, and Δ measured Log D can be extracted.

LIME scores provide sufficiently accurate explanations of Log D predictions for unseen molecules. Outliers are shown schematically as circled points on FIG. 5.

All references cited herein are incorporated by reference in their entireties.

The foregoing description is intended to illustrate various aspects of the instant technology. It is not intended that the examples presented herein limit the scope of the appended claims. The invention now being fully described, it will be apparent to one of ordinary skill in the art that many changes and modifications can be made thereto without departing from the spirit or scope of the appended claims. It is further to be understood that the appended claims are representative of several of the various embodiments described herein, and that any embodiment so described but not expressed in one of the appended claims may be expressed in a claim in an application claiming benefit of priority to the instant application without any concomitant loss of priority.

What is claimed:

1. A method, comprising:
receiving test molecular structure data for a test molecule, wherein the molecular structure data for the test molecule comprises an atom type for each atom in the test molecule;
inputting the test molecular structure data into a global model of a physicochemical property, wherein the global model comprises a contribution of each of a plurality of atom types to a value of the physicochemical property for the molecule, and wherein the global model was trained using a set of training molecules for which the value of the physicochemical property was known from experimental measurement;
generating a local model of the physicochemical property, wherein the local model is based on molecules in the neighborhood of the test molecule and wherein the neighborhood is defined according to a threshold value of a similarity metric;
optimizing the local model according to one or more best-fit criteria, thereby creating a best-fit local model;
validating the best-fit local model by:
using a match-pairs analysis to establish a set of molecules related to the test molecule by a set of respective chemical transformations;
obtaining from the best-fit local model weighted contributions to the physicochemical property of atoms and functional groups in the test molecule and atoms and functional groups in the set of molecules related to the test molecule;
for each molecule in the set of molecules related to the test molecule, calculating a first delta, wherein the first delta is the difference between the value of the sum of the weighted contributions of the one or more atoms in the chemical transformation of the molecule and value of the sum of the weighted contributions of the one or more atoms in the chemical transformation for the test molecule;
for each molecule in the set of molecules related to the test molecule, calculating a second delta, wherein the second delta is the difference between the value of the physicochemical property calculated in the global model for the molecule and the value of the physicochemical property calculated in the global model for the test molecule;
and
deriving from the values of the first delta and the values of the second delta the validity of an interpretability model for the physicochemical property wherein the interpretability model comprises weighted contributions of atoms and functional groups for a molecule in the set of molecules related to the test molecule to the value of the physicochemical property for the molecule.

2. The method of claim 1, wherein the deriving comprises plotting the values of the first delta against the values of the second delta for each of the molecules in the set of molecules related to the test molecule.

3. The method of claim 1, wherein the similarity metric is a Tanimoto coefficient or a cosine similarity coefficient.

4. The method of claim 1, wherein the similarity metric is derived from 1-dimensional representations of the molecules.

5. The method of claim 1, wherein the similarity metric is derived from 2-dimensional representations of the molecules.

6. The method of claim 1, wherein the similarity metric is derived from 3-dimensional representations of the molecules.

7. The method of claim 1, wherein the physicochemical property is log D.

8. The method of claim 1, wherein the physicochemical property is kinetic solubility.

9. The method of claim 1, wherein the physicochemical property is a weighted combination of kinetic solubility and log D.

10. The method of claim 1, wherein the test molecule comprises from 10-50 non-hydrogen atoms.

11. The method of claim 1, wherein the best-fit criterion is calculated according to a method selected from: linear regression and least squares.

12. The method of claim 1, wherein matched-pairs analysis is such that a molecule is similar to the test molecule if it differs from the test molecule by a single chemical transformation.

13. The method of claim 12, wherein the single chemical transformation is selected from the group consisting of: substituting one atom for another; substituting an atom for a functional group; inserting a single atom; and inserting a functional group.

14. The method of claim 1, wherein the validity is quantified by the coefficient of determination R².

15. The method of claim 1, wherein each atom type for a given atom is represented as a vector of weighted contributions of atoms in the functional group in which the atom is situated.

16. The method of claim 15, wherein the vector comprises values of properties selected from the group consisting of: atomic number, hybridization, number of neighbors, aromaticity.

17. The method of claim 15, wherein the vector of weighted contributions for an atom comprises contributions from up to 6 atoms, at least 2 of which are bonded to the atom.

18. The method of claim 1, wherein the validity is quantified by the Pearson correlation coefficient.

19. A computer-readable medium programmed with instructions for carrying out the method of claim 1.

20. A computing apparatus comprising one or more processors configured to execute a computer program for carrying out the method of claim 1, and for communicating output of the method to a user able to interpret the same.
